(19) United States
(12) Patent Application Publication
(10) Pub. No.: US 2011/0257762 A1
(43) Pub. Date: Oct. 20, 2011

(54) PROSTHETIC IMPLANT WITH A DELAYED BONDING EFFECT

(75) Inventors: Christian Mai, Lyon (FR); Laure-Cecile Martin, Temay (FR); Jean-Jacques Sala, Lyon (FR)

(73) Assignee: Compagnie De Recherche en Composants, Implants et Materiels Pour L'Application Clinique, Vienne (FR)

(21) Appl. No.: 13/120,809

(22) PCT Filed: Sep. 24, 2009
(86) PCT No.: PCT/EP2009/062351

§ 371 Date: Jul. 5, 2011

(30) Foreign Application Priority Data
Sep. 26, 2008 (JP) FR0805324

(51) Int. Cl. A61F 2/02

(52) U.S. Cl. 623/23.72; 427/2.24

(57) ABSTRACT

The invention relates to a prosthetic system (1) that can implanted in a human or animal body, wherein said system (1) includes a substrate (2) to be brought into contact with a biological tissue (4), and an adhesive (3) for bonding said substrate (2) onto the biological tissue (4), said system (1) being characterised in that the adhesive (3) contains as an adhesive substance at least one hydrocolloid. The invention can be used for prosthetic implants.

The present invention relates to the overall technical field of implantable systems used, in particular, in human or animal surgery.

The present invention relates more specifically to an implantable prosthetic system that can be implanted in a human or animal body, said system comprising firstly a support intended to come into contact with a biological tissue and secondly an adhesive intended to cause said support to adhere to the biological tissue.

The present invention also relates to a prosthetic implant comprising an implantable prosthetic system in accordance with the foregoing.

The present invention further relates to a method of manufacturing an implantable prosthetic system, said method comprising, firstly, a step of providing a support intended to come into contact with a biological tissue, and secondly a step of manufacturing an adhesive intended to cause said support to adhere to the biological tissue.

The present invention furthermore relates to an adhesive intended to cause a support to adhere to a biological tissue.

And finally, the present invention relates to a method of manufacturing an adhesive intended to cause a support to adhere to a biological tissue.

It is known practice to resort to implants, notably textile prosthesis, for treating hernias or eventrations. These implants are generally held in contact with the hernia or with the eventration in order to provide effective treatment.

Conventionally, fastening means of the clip type are used to hold the implants in position. These clips, although providing a dependable way of attaching the implant to the tissues, carry not insignificant risks of causing lesions in the tissues and of causing the patient pain, notably because of their long-term presence within the patient's body and because of the risk that they might touch a nerve ending. Further, the use of these clips is particularly expensive and entails equipment that is single-use equipment and likewise expensive.

In order to address the problems associated with the use of these clips, resorbable clips have been developed which break down within the body after a set and adaptable time of use. The use of these resorbable clips, although it does reduce the risk of lesions and chronic pain for the patient, tends to be expensive and to require complex and expensive fitment devices. Further, such resorbable clips do not limit the risk of lesions and pain in the short term.

In order to alleviate the disadvantages of the use of clips, resorbable or otherwise, for attaching implants in the context of the treatment of hernias and eventrations, it has been proposed to use adhesives which allow the implant to be bonded directly to a biological tissue, without the intervention of positive means of attachment that bite into the tissues. In general, the surgeon coats the implant with adhesive before introducing the implant into the region of the hernia or of the eventration. The adhesive is therefore applied in situ once the ingredients of which it is composed have been mixed.

Biological adhesives, for example fibrin adhesive derived from human blood plasma, are particularly well known. Although this adhesive yields good results, its use remains highly limited because of the risks of contamination that it carries.

It is also known practice to use adhesives based on cyanoacrylate, which limit the risks of contamination. However, these adhesives have the disadvantage of often being particularly harmful to the patient because of their aggressiveness toward the tissue and their lack of long-term biocompatibility.

The stated objectives of the present invention are therefore to address the aforementioned disadvantages and to propose a new implantable prosthetic system that is simple and quick to implement, and the bonding of which is practical and effective.

Another stated objective of the invention is to provide a new implantable prosthetic system that is well tolerated by the patient and carries no risk of rejection by or pain to the patient.

Another stated objective of the invention is to propose a new implantable prosthetic system the bonding of which is reliable, stable and ready for use.

Another stated objective of the invention is to propose a new implantable prosthetic system the bonding of which limits the risk of undesirable shifting or unwanted detachment of the system.

Another stated objective of the invention is to propose a new implantable prosthetic system the composition of which allows it to hold firmly on the biological tissue.

Another stated objective of the invention is to provide a new implantable prosthetic system that can be simply and quickly repositioned on the biological tissue.

Another stated objective of the invention is to provide a new implantable prosthetic system the composition of the adhesive of which improves the qualities and properties of the bonding of the system when it comes into contact with the biological tissue.

Another stated objective of the invention is to provide a new implantable prosthetic system which contains ingredients which are known, readily available, low in cost and particularly biocompatible.

Another stated objective of the invention is to provide a new implantable prosthetic system the shape of which is adapted to suit its use in the human or animal body, and makes it easier to bring into contact with the biological tissue.

Another stated objective of the invention is to provide a new implantable prosthetic system that encourages healing in a simple and effective way.

Another stated objective of the invention is to provide a new implantable prosthetic system that can be used for treating a hernia or an eventration.

Another stated objective of the invention is to provide a new prosthetic implant that is easy and quick to implement, and the bonding of which is practical and effective.

Another stated objective of the invention is to provide a new method of manufacturing an implantable prosthetic system that is simple and quick to perform, is inexpensive, and uses ingredients that are known and readily available.

Another stated objective of the invention is to provide a new method of manufacturing a prosthetic implant that is simple and quick to implement, making it possible to obtain an implant that is simple to use and well tolerated by the patient.

Another stated objective of the invention is to provide a new adhesive that is easy and quick to implement and the use of which is practical and effective.

Another stated objective of the invention is to provide a new adhesive that is well tolerated by the patient and carries no risk of rejection by or pain to the patient.

Another stated objective of the invention is to provide a new adhesive, ready for use, the bonding of which is reliable, stable and limits the risk of undesirable shifting or unwanted detachment of the system.

Another stated objective of the invention is to provide a new adhesive which uses ingredients that are known, low cost, available and biocompatible.

Another stated objective of the invention is to provide a new adhesive which has good properties of bonding upon contact with the support and/or the biological tissue.

Another stated objective of the invention is to provide a new adhesive which is simple and effective to implant, and for this to be brought into contact with the biological tissue in a particularly easy and adjustable way.

Another stated objective of the invention is to provide a new method of manufacturing an adhesive that is simple and quick to perform, is inexpensive, and uses ingredients that are known and readily available.

Another stated objective of the invention is to provide a new method of manufacturing an adhesive that involves production steps that are simple, and yield an adhesive that is effective and easy to use.

Another stated objective of the invention is to provide a new surgical method of implanting an implantable prosthetic system which is easy and quick to implement and which limits pain to the patient and post-operative complications.

Another stated objective of the invention is to provide a new surgical method of implanting an implantable prosthetic system which makes the surgeon's actions easier while at the same time allowing the surgeon to adapt the prosthetic system at will.

The stated objectives of the invention are achieved using an implantable prosthetic system that can be implanted in a human or animal body, said system comprising firstly a support intended to come into contact with a biological tissue and secondly an adhesive intended to cause said support to adhere to the biological tissue, said system being characterized in that said adhesive comprises, by way of adhesive substance, at least one hydrocolloid.

The stated objectives of the invention are also achieved using a prosthetic implant comprising an implantable prosthetic system in accordance with the foregoing, in which the adhesive is attached to the support by construction.

The stated objectives of the invention are furthermore achieved using a method of manufacturing an implantable prosthetic system, said method comprising, firstly, a step of providing a support intended to come into contact with a biological tissue, and secondly a step of manufacturing an adhesive intended to cause said support to adhere to the biological tissue, said method being characterized in that the step of manufacturing the adhesive comprises a substep of providing at least one hydrocolloid used in the adhesive by way of adhesive substance.

The stated objectives of the invention are likewise achieved using an adhesive intended to cause a support to adhere to a biological tissue, said adhesive being characterized in that it contains, by way of adhesive substance, at least one hydrocolloid.

The stated objectives of the invention are furthermore achieved using a method of manufacturing an adhesive intended to cause a support to adhere to a biological tissue, said method being characterized in that it comprises a step of manufacturing the adhesive, during which step at least one hydrocolloid is supplied by way of adhesive substance.

The stated objectives of the invention are finally achieved using a surgical method of implanting an implantable prosthetic system, said system comprising firstly a support intended to come into contact with a biological tissue, and secondly an adhesive intended to cause said support to adhere to the biological tissue, said method first of all comprising a step of introducing said system into the body, then a step of bonding said support to the biological tissue, during which step at least one hydrocolloid is used by way of adhesive substance.

Further objects, features and advantages of the invention will become apparent in further detail from reading the following description and from the attached drawings which are provided purely by way of nonlimiting illustration and in which:

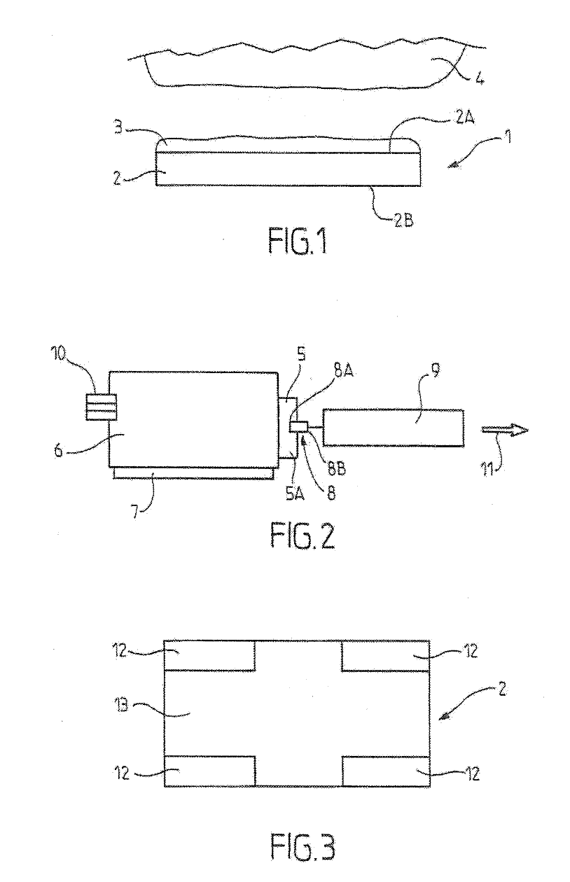

FIG. 1 illustrates, in a view in cross section, a prosthetic system according to the invention.

FIG. 2 illustrates, in a schematic view from above, a bonding test in which a prosthetic system according to the invention is held between two pieces of meat.

FIG. 3 depicts, in a schematic view from above, a prosthetic system according to the invention in which the support comprises an adhesive and a cell colonization activator.

The present invention relates to an implantable prosthetic system 1 that can be implanted into a human or animal body. The system 1 of the invention is therefore specifically designed to be able to be implanted, that is to say introduced, inside a human or animal organism, so as to be completely encompassed by said organism, with no contact with the outside, on completion of the surgical implantation operation.

Within the meaning of the present invention, a system contains at least two independent elements which may or may not be joined together by construction. In this particular instance, the system 1 comprises firstly a support 2 intended to come into contact with a biological tissue 4 and secondly an adhesive 3 intended to cause said support 2 to adhere to the biological tissue 4.

For preference, the support 2 is of particularly supple nature so that it can easily be implanted in the body of the patient and perfectly follow the shapes and movements of the biological tissues 4 adjacent to said support. For preference, the support 2 is manufactured from a strong material that is well tolerated by the patient and limits the risks of infection or rejection. The support 2 is advantageously made of a biocompatible textile material, for example polypropylene.

In this case, the prosthetic system 1 constitutes a parietal prosthesis for treating a hernia or an eventration, said prosthesis allowing the abdominal wall to be strengthened by notably closing a puncture or hole in the abdominal wall. Alternatively, the support 2 may consist of some other supple textile implantable prosthetic system 1. Thus, for example, it may constitute a suburethral tape designed to support the urethra to treat urinary incontinence. It is also possible for the system of the invention to form a colposuspension prosthesis for treating a genital prolapse, although other surgical applications are also conceivable.

Alternatively, the support 2 may be made of a material of the polymer type and used, for example, as a silicone-implantable site or as a heart valve. Further, it is also conceivable for the support 2 to be made of a metallic and/or ceramic material, it then being possible for said support 2 for example to be a hip prosthesis, a dental implant, or a dental appliance.

An implantable prosthetic system 1 such as this can thus be used in numerous applications, for example as a parietal support, as a means of attachment, a means of retention, a means of support or a means of reinforcement. The shape, size and material of the support 2 can therefore vary according to the destination of the system 1 and the end-use to which it is put.

To make the system of the invention 1 easier to understand, one particularly advantageous embodiment of the implantable prosthetic system 1 will be described in the remainder of the description and in this particularly advantageous embodiment the system forms a prosthesis for reinforcing a wall or providing support, and is preferably a textile prosthesis. For preference, the system 1 is therefore designed to consolidate any type of wall or to support, hold or retain an organ, a wall or a muscle, within the organism.

Alternatively, it is quite obviously conceivable for the prosthetic system 1 of the invention to be designed for any other application.

The adhesive 3 of the present invention allows the support 2 to be made to adhere to the biological tissue 4, that is to say allows said support 2 to be fixed to the intracorporeal tissues 4 to which said support 2 is intended to be fixed. Advantageously, the adhesive 3 provides dependable attachment of said support 2 to the tissues 4 in the functional position, notably to the abdominal wall, said fixing being achieved in a way that is stable enough to last, without degradation and optimally, through the duration of the treatment. In other words, the support 2 is fixed or held in its functional position on the biological tissue using an adhesive 3, said adhesive 3 being of a composition that is compatible with the human body. The adhesive 3 and the support 2 may be independent or welded together. The adhesive 3 for example covers all or part of one of the faces 2A, 2B of said support 2.

Said adhesive 3 contains, by way of adhesive substance, at least one hydrocolloid. For preference, the hydrocolloid of the invention comprises particles of a size ranging between substantially one nanometer and one micrometer. Advantageously, said particles are not water soluble and in an aqueous medium form a homogeneous solution.

Advantageously, and despite the existence of earlier adhesives based on synthetic compounds which guarantee correct bonding, surprising bonding results, at least equivalent to or even appreciably better than those obtained with these earlier adhesives, have been obtained using an adhesive containing natural components. Thus, advantageously, the hydrocolloid of the invention is chosen from natural hydrocolloids, that is to say is of natural origin.

For preference, the natural hydrocolloid is of plant origin and is thus extracted from all or part of a plant. In this case, the hydrocolloid is derived for example from a fruit, a grain or from sap. Alternatively, the natural hydrocolloid is advantageously derived from bacterial fermentation.

In other words, the hydrocolloid is obtained naturally and has not been chemically synthesized. Such a natural hydrocolloid has the advantage of being well tolerated by the organism and limits the risks of intolerance that there might be with synthetic hydrocolloids. Further, natural hydrocolloids are readily available, perfectly biocompatible, and well known.

Advantageously, said hydrocolloid is intended to form at least one weak bond with the biological tissue 4 to bond said support 2 to said biological tissue 4. The adhesive function of the hydrocolloid of the invention is, in fact, obtained through the creation of weak bonds between said hydrocolloid and the tissue 4, notably between the hydrocolloid and the tissues 4 of the organism.

Within the meaning of the invention, the weak bonds are ionic bonds, hydrogen bonds or Van der Waals bonds which can form naturally between two molecules in the presence of one another, as opposed to strong bonds of the covalent bond type which generally require a chemical reaction. For preference, the hydrocolloid therefore encourages the adhesion of the support 2 to the biological tissue 4 when a plurality of weak bonds is established between the hydrocolloid and the tissue 4.

As a preference, said hydrocolloid contains at least one electrolyte group intended to form at least one weak bond with the biological tissue 4, so as to bond said support 2 to said biological tissue 4.

Within the present invention, an electrolyte group is considered preferably to be an ionic or ionizable group, namely a highly polar chemical grouping present within the molecule that makes up the hydrocolloid, said electrolyte being capable of forming weak bonds with the biological tissues 4 so as to allow the support 2 to bond with said tissues 4 in a reliable and stable manner.

It is preferable for said at least one hydrocolloid to be a polyelectrolyte hydrocolloid, that is to say a hydrocolloid containing a plurality of electrolyte groups which are intended to form weak bonds with the biological tissues 4. Within the meaning of the invention, the electrolyte groups are, for example, carboxyl groups and SO₃H groups.

The electrolyte groups of the hydrocolloids advantageously have a tendency to form weak bonds with the chemical compounds that make up the tissues 4 and are therefore, at least in part, responsible for the bonding effect that allows the support 2 to be bonded in a stable manner to the biological tissues 4.

Furthermore, said hydrocolloid allows said support 2 to adhere to the biological tissue 4 advantageously with a bonding force appreciably in excess of 5 Newtons (N), preferably appreciably in excess of 10 Newtons (N). Thus, the use of the weak bonds makes a very marked contribution toward the strength of the bond by notably making it possible to obtain a bonding force in excess of 10 N, namely a bonding force that is strong enough that it avoids any migration of the support once said support 2 has been laid on the tissue 4. Such a bonding force provides a stable, reliable and durable bond between the support 2 and the tissue 4 throughout the duration of the treatment.

The hydrocolloid of the invention further comprises other properties that encourage the bonding. Thus, it advantageously has a viscosity that is high enough to, in the presence of water, afford a bonding effect. The hydrocolloid is capable of absorbing the water present in the biological tissues 4, thus causing it to swell and become hydrated, at least in part. Consequently, in the presence of water, and notably upon contact with the tissue 4, the electrolyte groups of the hydrocolloid become accessible and establish weak bonds with the biological tissue 4, said bonds causing adhesion.

Further, the viscosity of the hydrocolloid encourages durable and reliable retention of the support 3 which does not migrate and which remains in position on the tissues 4. This property means that a prosthetic system 1 can be obtained with an adhesive 3 which remains on the support 2, does not run and does not dissolve in the presence of the biological tissues 4, said adhesive 3 thus allowing said support 2 to adhere effectively to the tissues 4.

Furthermore, the hydrocolloid becomes hydrated slowly, in the presence of water, notably upon contact with the biological tissues 4, in order to afford a delayed bonding effect.

Specifically, the weak bonds, which for the most part become created after water of the tissues 4 has entered the molecule of the hydrocolloid, also forms slowly, and this gives rise to delayed-action bonding of said support 2 to the tissues 4.

Such a delayed bonding effect, caused mainly by the viscosity of the hydrocolloid and the time it takes to become hydrated, allows the surgeon who is implanting the prosthetic system 1 the option of repositioning said system 1 before it becomes definitively bonded to the biological tissue 4. Specifically, before, the bonding of the support 2 to the biological tissue 4 becomes truly effective, the surgeon has a bit of time, the length of which depends on the viscoelastic and hydration properties of the hydrocolloid, in which if necessary to modify the positioning of the system 1, for example to position said system 1 to best effect.

In order to provide an effective solution to the desired mechanical properties like those mentioned hereinabove, the adhesive 3 contains certain hydrocolloids in particular. For preference, the adhesive 3 contains one or more of the following hydrocolloids: carrageenan, gum tragacanth, pectin, gellan gum, xanthan gum, gum arabic and guar gum. These natural hydrocolloids have bonding forces appreciably in excess of 5 N and, for the most part, in excess of 10 N. These are generally hydrocolloids which are well known, readily accessible and, for the most part, used in the agrifoodstuffs industry and therefore particularly well tolerated. It is also conceivable to use other hydrocolloids if they have the properties mentioned hereinabove and essential for effective and practical bonding.

Advantageously, the adhesive contains one or more hydrocolloids, namely it is possible for the adhesive to contain a mixture of several of the abovementioned hydrocolloids in proportions that can vary according to the expected bonding properties.

The bonding properties of these various hydrocolloids have also been demonstrated using a test described hereinbelow and illustrated in FIG. 2.

Test

Objective

The object of this test was to quantify the detachment force, under shear, of a prosthetic system 1 according to the invention, in which each of the aforementioned hydrocolloids is tested.

Method and Equipment

Six sheets of textile 5 of identical size and material, were each coated with one of the following hydrocolloids, said hydrocolloid being spread in identical and uniform quantities over each of the six sheets 5: carrageenan, gum tragacanth, pectin, gellan gum, xanthan gum, gum arabic and guar gum.

As can be seen in FIG. 2, each coated sheet 5 was placed between two pieces of meat 6, 7 to simulate the tissues of the human body, for example between two steaks, said steaks 6, 7 being of identical size, shape and weight. The sheet 5 was positioned between the steaks 6, 7 so that a portion 5A of the sheet 5 protrudes so that it can easily be pulled in order to take measurements during the bonding test. Pressure was applied to improve the bond. The sheet 5 was then left between the two steaks 6, 7 for a resting time of several hours, preferably between 3 and 20 hours, the resting times being perfectly identical for all six sheets 5.

After the resting time, a first end 8A of a crocodile clip 8 was attached to that portion of the sheet 5A that was protruding, and a second end 8B of the crocodile clip 8 was attached to a dynamometer 9. A retaining device 10 held the steaks 6, 7 assembly throughout the duration of the test.

A force was applied to the portion 5A of the sheet, in the direction of the arrow 11, in an attempt to detach the sheet 5A from the steaks 6, 7. The dynamometer 9 measured the shear force needed to detach the sheet 5 from the steaks 6, 7. The operation was repeated for each one of the six sheets.

Results

The results of the shear detachment forces obtained for the various hydrocolloids are collated in the following table:

| | Detachment force in Newtons |
|---|---|
| Hydrocolloid | (N) |
| Carrageenan | 21.5 |
| Gum tragacanth | 14.5 |
| Pectin | 16.0 |
| Gellan gum | 35.0 |
| Xanthan gum | 13.7 |
| Guar gum | 7 |

The results show that the hydrocolloids of the invention yield bonding forces in excess of 5 N, preferably in excess of 10 N. The bonding of the support 2 to a tissue 4 is considered to be sufficient when the bonding force is at least between 10 and 15 N. In this particular case, it can be concluded that carrageenan, gum tragacanth, pectin, gellan gum and xanthan gum provide effective and reliable bonding of the prosthetic system 1 with bonding forces in excess of 10 Newtons. These hydrocolloids are polyelectrolytes.

Guar gum, which is not a polyelectrolyte, gives a bonding force of 7 N which is markedly lower than that of the polyelectrolyte hydrocolloids. Consequently, it can be concluded that the presence of several electrolyte groups in the hydrocolloid improves the bond. Further, it is then possible, depending on the bonding strength desired, to adapt the composition of the adhesive by mixing together several hydrocolloids that have different bonding forces.

This test thus shows the surprising and particularly advantageous effect of using polyelectrolyte hydrocolloids for bonding an implantable prosthetic system 1 in a way that is stable, reliable and optimal throughout the duration of the treatment.

In addition to containing these hydrocolloids, the adhesive 3 may advantageously contain other ingredients able to contribute to improving the support 2 bonding effect. It is thus conceivable, in one particularly advantageous embodiment, for said adhesive 3 to contain at least one calcium salt and/or one sodium salt and/or one magnesium salt and/or one potassium salt, said salt being intended to modify the viscoelastic properties of said at least one hydrocolloid, to encourage bonding. For preference, said at least one salt is intended, in the presence of water, to release calcium and/or sodium and/or magnesium and/or potassium ions, said ions being intended to encourage said hydrocolloid to gel.

In other words, a salt which will have a tendency to dissolve in the presence of water upon contact of the support 2 with the biological tissues 4 is added to the adhesive 3 which is made up mainly of one or more hydrocolloids as defined hereinabove. The dissolving of this salt gives rise to the release of cations, particularly calcium Ca²⁺ and/or sodium Na²⁺ and/or magnesium Mg²⁺ and/or potassium K⁺ ions, these ions being intended to encourage gelling. Specifically, these ions allow the hydrocolloids to gel by creating bridges, that is to say weak bonds, between the electrolyte groups of said hydrocolloid, so as to create a physical gel. These ions therefore advantageously densify the hydrocolloid structure by gelling it. The gel formed by the addition of these ions has a tendency to encourage bonding by creating a network of weak bonds between the electrolyte groups of the hydrocolloid, on the one hand, and between the electrolyte groups of the hydrocolloid and the tissue 4, on the other.

Advantageously, the implantable prosthetic system 1 of the invention is in a layered form, said adhesive 3 being made up of at least two substantially superposed layers, each of the layers containing one or more hydrocolloid(s) chosen from: carrageenan, gum tragacanth, pectin, gellan gum, xanthan gum, gum arabic and guar gum. Thus, in this advantageous embodiment, the adhesive 3 is made up of several layers, for example coats, pellets or spots, laid on top of one another.

Having the adhesive 3 configured in this way encourages good control over the viscosity and hydration of said adhesive 3 at the time it comes into contact with the tissues 4 and thus provides control over the bonding effect. Specifically, the adhesive 3 may become hydrated at a greater or lesser rate upon contact between the first layer of hydrocolloid with the tissue 4, hydration then potentially speeding up or slowing down as water from the tissues enters the second layer of the adhesive 3. The use of an adhesive 3 arranged in layers thus encourages control over the bonding of the support to the biological tissue 4.

Moreover, said adhesive 3 of the invention may advantageously contain at least one cell colonization activator compound, preferably collagen or a hyaluronic acid. In other words, an ingredient that stimulates cell growth, namely which encourages the tissue 4 to heal, is added to the composition of the adhesive. The hyaluronic acid and/or the collagen is used to speed the development of cells in the biological tissues 4. These compounds are well known to those skilled in the art, are effective, are readily available and are simple to use.

It is also possible for said support 2 to be covered, at least in part, with such a cell colonization activator compound. As has been illustrated in FIG. 2, said support 2 comprises regions 12 which are coated with hydrocolloids and a region 13 which is coated with one or more cell growth activators. In this advantageous embodiment, the support 2 is held on the biological tissues 4 by the adhesive regions 12 and allows said tissues 4 to heal using the cell growth activating region 13 which also comes into contact with said tissues 4.

The composition of the adhesive 3 thus makes it possible to obtain a prosthetic system 1 which has both optimal bonding properties and good healing properties.

As a preference, the adhesive 3 is impregnated into all or just part of the support 2 and is then dried thereon. For preference, said adhesive 3 is freeze-dried, that is to say undergoes a conventional freeze-drying process on completion of which it is in a completely dehydrated form on the support 2. Having the adhesive 3 in this state contributes toward appreciably improving the suppleness or flexibility of the prosthetic system 1 and toward making it easier for the surgeon to use, notably avoiding the somewhat impractical effect of immediate adhesion of liquid adhesives that have to be coated onto the support prior to implanting it. Specifically, such adhesives are also impractical and complicated to use because they have to be applied to the implant during the course of the surgical operation, and this tends to make the operation more complicated, take longer, and cost more.

The invention also covers, as such, a prosthetic implant comprising an implantable prosthetic system 1 according to the above description, in which the adhesive 3 is attached to the support 2 by construction. Advantageously, such an implant therefore comprises, as a single piece, namely substantially as a single entity, a support 2 and an adhesive 3. For preference, the implant is obtained by impregnating or spraying adhesive 3 over all or just part of the support 2, followed by drying.

Furthermore, the present invention relates to a method of manufacturing an implantable prosthetic system 1. This method comprises, first, a step of supplying a support 2 intended to come into contact with a biological tissue 4. The support 2 is manufactured in accordance with a conventional method for manufacturing a prosthesis made of textile or any other implantable device as defined hereinabove. One method of manufacturing a prosthesis for reinforcing a wall or providing a support, for example a parietal prosthesis used in the treatment of hernias and eventrations will be described by way of preference.

The method secondly comprises a step of manufacturing an adhesive 3 intended to cause said support 2 to adhere to the biological tissue 4, said adhesive 3 being potentially in accordance with the foregoing. The step of manufacturing the adhesive 3 comprises a substep of providing at least one hydrocolloid used by way of adhesive substance in the adhesive 3. For preference, a hydrocolloid is chosen from the aforementioned ones, namely chosen from: carrageenan, gum tragacanth, pectin, gellan gum, xanthan gum, gum arabic and guar gum.

For preference, after the two steps of supplying the support 2 and of manufacturing the adhesive 3, the method comprises a step of assembling said adhesive 3 with said support 2 so as to obtain a one-piece implantable prosthetic system 1. Advantageously, the step of assembling said adhesive 3 with said support 2 comprises a substep of impregnating said support 2 with a liquid matrix containing said at least one hydrocolloid so as to cause said at least one hydrocolloid to adhere to the support 2. For preference, said adhesive covers all or part of said support 2, it being possible for the impregnation of the support 2 to be partial or total.

Indeed, in a preferred embodiment of the invention, a liquid matrix containing the adhesive 3, made up for the most part of at least one hydrocolloid, is prepared. The support 2 is then dipped in said liquid matrix so that the adhesive 3 can impregnate the support 2.

It is also possible to add other ingredients to the composition of the adhesive. Thus, for preference, the step of manufacturing said adhesive 3 comprises a substep of mixing said at least one hydrocolloid with at least one calcium salt and/or one sodium salt and/or one magnesium salt and/or one potassium salt, said salt being intended to modify the viscoelastic properties of said at least one hydrocolloid, so as to encourage bonding.

Advantageously, a liquid matrix is prepared which contains both the hydrocolloid and the salt. Said support 2 is then immersed in the liquid matrix so that the latter becomes perfectly and uniformly applied to said support 2, either over the entirety of the support 2 or over just part of said support 2. Said support 2 is then finally dried, for example by traditional drying in an oven or by freeze-drying the liquid matrix onto the support 2.

This method makes it possible to obtain an implantable prosthetic system 1 which carries on board it an adhesive 3 which is ready for use. This method also has the advantage of yielding a system 1 that is easy for a surgeon to use as the surgeon then does not have to coat the support 2 himself before implanting the system 1 in the human body.

The present invention also relates as such to an adhesive 3 intended to cause a support 2 to adhere to a biological tissue 4. Said adhesive 3 of the invention comprises, by way of adhesive substance, at least one hydrocolloid. By way of adhesive 3 must be understood to mean an element capable of fixing two surfaces together, the two surfaces in this instance being a support 2 and a biological tissue 4, the adhesive 3 preferably being an adhesive like the adhesive 3 previously described.

Advantageously, said at least one hydrocolloid is chosen from natural hydrocolloids. This hydrocolloid is advantageously intended to form at least one weak bond with the biological tissue 4 in order to bond said support 2 to said biological tissue 4.

Advantageously, said hydrocolloid comprises at least one electrolyte group intended to form at least one weak bond with the biological tissue 4 so as to bond said support 2 to said biological tissue 4.

Furthermore, the adhesive 3 of the invention advantageously comprises one or more of the following hydrocolloids: carrageenan, gum tragacanth, pectin, gellan gum, xanthan gum, gum arabic and guar gum. These hydrocolloids are, as has already been explained, chosen for their properties, notably for their viscoelasticity, their natural origin, their molecular weight, the number of electrolyte groups they have and their availability, this list not being exhaustive.

In one particularly advantageous embodiment of the invention, the adhesive 3 also comprises at least one calcium salt and/or one sodium salt and/or one magnesium salt and/or one potassium salt, said salt being intended to modify the viscoelastic properties of said at least one hydrocolloid, to encourage bonding. For preference, said at least one salt is intended, in the presence of water, to release calcium and/or sodium and/or magnesium and/or potassium ions, said ions being intended to encourage said hydrocolloid to gel.

The addition of such a salt into the composition of the adhesive 3 has the benefit of appreciably improving the gelling of the hydrocolloid and thus encouraging bonding.

It is preferable for said adhesive 3, made up predominantly of at least one hydrocolloid, to be dried so that it is present in functionally optimal form. In such a case, said at least one hydrocolloid is advantageously freeze-dried.

What is more, the present invention relates to a method of manufacturing an adhesive 3 intended to cause a support 2 to adhere to a biological tissue 4. The method comprises a step of manufacturing the adhesive 3, during which step at least one hydrocolloid is provided by way of adhesive substance. During this step of manufacturing the adhesive 3, one or more hydrocolloids chosen from those already mentioned are placed in in a liquid matrix.

Advantageously, this manufacturing, step comprises a substep of mixing said at least one hydrocolloid with at least one calcium salt and/or one sodium salt and/or one magnesium salt and/or one potassium salt, said salt being intended to modify the viscoelastic properties of said at least one hydrocolloid.

This substep of mixing has the benefit of bringing into the composition of the adhesive 3 an ingredient that provides a significant improvement in the gelling and therefore the adhesion of the hydrocolloid.

The method of manufacturing the adhesive comprises, after the step of manufacturing the adhesive 3, a step of forming the adhesive 3 into a substantially flat film. During this forming step, the adhesive 3 is given such a configuration that it can easily be used, notably in surgery, for the internal bonding of a support 2 to a biological tissue 4. For preference, the goal is to obtain an adhesive 3 of flattened shape, small thickness, that is to say in a format substantially identical to that of the bandages or compresses commonly used for treating surface wounds, the adhesive 3 being perfectly encompassed inside the body of the patient and used for internal bonding not visible from the outside.

Finally, the method comprises, after the forming step, a step of drying said adhesive 3 by freeze-drying so as to obtain a substantially supple adhesive 3. During this step, the adhesive 3 is freeze-dried and the end of the method yields an adhesive in the form of a film or sheet which is substantially supple and can be used directly inside the human body.

This method, which is simple and quick to implement, makes it possible to obtain an adhesive 3 the composition and format of which encourage simple use for easier, durable and dependable bonding.

Finally, and independently of the foregoing, the present invention relates to a surgical method for implanting an implantable prosthetic system, said system comprising firstly a support 2 intended to come into contact with a biological tissue 4 and secondly an adhesive 3 intended to cause said support 2 to adhere to the biological tissue 4. To make the method easier to understand, a surgical method of implanting a prosthesis that strengthens a wall or provides support, for example a parietal prosthesis for treating an eventration will be described.

Said method first of all comprises a step of introducing said system 1, namely the prosthesis, into the body, during which step the surgeon implants said system 1 in the patient's body. Said prosthesis is a prosthesis in accordance with the foregoing and at its surface comprises an on-board adhesive 3, in substantially dehydrated form and ready for use.

Next, said method comprises a step of bonding said support 2 to the biological tissue 4, during which step at least one hydrocolloid is used by way of adhesive substance. During the bonding step, the surgeon positions the parietal prosthesis that acts as a support in the desired position. During the bonding step, said hydrocolloid is preferably intended to form at least one weak bond with the biological tissue 4, so as to bond said support 2 to the biological tissue 4.

Furthermore, advantageously, during the bonding step, the at least partial hydration of the adhesive 3 upon contact with the tissues is slow and thus allows the surgeon easily to reposition the system 1 before the bonding becomes more or less definitive. Specifically, the presence of at least one hydrocolloid in the adhesive 3 has a tendency to delay the bonding effect. The surgeon therefore has a bit of time, of varying length, in which to reposition the prosthesis until he finds its functionally optimal position, the length of said time being at least partially dependent on the nature of the hydrocolloids used.

During the step of bonding said prosthesis, a surgeon may, if he so wishes, accelerate the bonding by applying pressure to the prosthesis. Such pressure encourages the hydration of the hydrocolloid, thus speeding the creation of weak bonds and speeding adhesion.

Such a surgical method therefore has the benefit of being easy and quick to implement. It also makes the operation of bonding a prosthesis in position far easier and avoids the surgeon having to perform lengthy and tricky actions.

1-32. (canceled)

33. A prosthetic implant comprising an implantable prosthetic system (1) that can be implanted in a human or animal body to form a prosthesis that reinforces a wall or provides support, said system (1) comprising firstly a support (2) intended to come into contact with a biological tissue (4), and secondly an adhesive (3) attached to the support by construction and intended to cause said support (2) to adhere to the biological tissue (4), said system (1) being characterized in that said adhesive (3) comprises, by way of adhesive substance, at least one hydrocolloid.

34. The prosthetic implant as claimed in claim 33, characterized in that said hydrocolloid is chosen from natural hydrocolloids.

35. The prosthetic implant as claimed in claim 33, characterized in that said hydrocolloid is intended to form at least one weak bond with the biological tissue (4) so as to bond said support (2) to said biological tissue (4).

36. The prosthetic implant as claimed in one of claim 33, characterized in that said hydrocolloid comprises at least one electrolyte group intended to form at least one weak bond with the biological tissue (4), so as to bond said support (2) to said biological tissue (4).

37. The prosthetic implant as claimed in claim 36, characterized in that said at least one hydrocolloid is a polyelectrolyte hydrocolloid.

38. The prosthetic implant as claimed in claim 33, characterized in that said hydrocolloid allows said support (2) to adhere to the biological tissue (4) with a bonding force appreciably in excess of 5 Newtons, preferably appreciably in excess of 10 Newtons.

39. The prosthetic implant as claimed in claim 33, characterized in that the hydrocolloid becomes hydrated slowly in the presence of water, in order to afford a delayed bonding effect.

40. The prosthetic implant as claimed in claim 33, characterized in that said adhesive (3) contains at least one calcium salt and/or one sodium salt and/or one magnesium salt and/or one potassium salt, said salt being intended to modify the viscoelastic properties of said at least one hydrocolloid, to encourage bonding.

41. The prosthetic implant as claimed in claim 40, characterized in that said at least one salt is intended to, in the presence of water, release calcium and/or sodium and/or magnesium and/or potassium ions, said ions being intended to encourage said hydrocolloid to gel.

42. The prosthetic implant as claimed in claim 33, characterized in that the adhesive (3) contains one or more of the following hydrocolloids: carrageenan, gum tragacanth, pectin, gellan gum, xanthan gum, gum arabic and guar gum.

43. The prosthetic implant as claimed in claim 33, characterized in that said adhesive (3) is freeze-dried.

44. The prosthetic implant as claimed in claim 33, characterized in that it is in stratified form, said adhesive (3) being made up of at least two substantially superposed layers, each of the layers containing one or more hydrocolloid(s) chosen from: carrageenan, gum tragacanth, pectin, gellan gum, xanthan gum, gum arabic and guar gum.

45. The prosthetic implant as claimed in claim 33, characterized in that said adhesive (3) contains at least one cell colonization activator compound.

46. A method of manufacturing a prosthetic implant comprising an implantable prosthetic system (1), said method comprising, firstly, a step of providing a support (2) intended to come into contact with a biological tissue (4), and secondly a step of manufacturing an adhesive (3) intended to cause said support (2) to adhere to the biological tissue (4), said method being characterized in that the step of manufacturing the adhesive (3) comprises a substep of providing at least one hydrocolloid used in the adhesive (3) by way of adhesive substance.

47. The method of manufacturing a prosthetic implant comprising an implantable prosthetic system (1) as claimed in claim 46, characterized in that the step of manufacturing said adhesive (3) comprises a substep of mixing said at least one hydrocolloid with at least one calcium salt and/or one sodium salt and/or one magnesium salt and/or one potassium salt, said salt being intended to modify the viscoelastic properties of said at least one hydrocolloid, so as to encourage bonding.

48. The method of manufacturing a prosthetic implant comprising an implantable prosthetic system (1) as claimed in claim 46, characterized in that it comprises a step of assembling said adhesive (3) with said support (2) so as to obtain a one-piece implantable prosthetic system (1).

49. The method of manufacturing a prosthetic implant comprising an implantable prosthetic system (1) as claimed in claim 48, characterized in that the step of assembling said adhesive (3) with said support (2) comprises a substep of impregnating said support (3) with a liquid matrix containing said at least one hydrocolloid, so as to cause said at least one hydrocolloid to adhere to the support (2).
